(19) United States
(12) Patent Application Publication
(10) Pub. No.: US 2026/0047764 A1
(43) Pub. Date: Feb. 19, 2026

(54) METHOD FOR IDENTIFYING INTRAVASCULAR PARTICLES USING ACOUSTIC ANALYSIS

(71) Applicant: Gwangju Institute of Science and Technology, Gwangju (KR)

(72) Inventor: Hae Min KIM, Gwangju (KR)

(21) Appl. No.: 19/299,904

(22) Filed: Aug. 14, 2025

(30) Foreign Application Priority Data
Aug. 16, 2024 (KR) 10-2024-0110092

(51) Int. Cl. A61B 5/00; A61B 5/145

(52) U.S. Cl. CPC A61B 5/0095; A61B 5/004; A61B 5/14546; A61B 2576/02

(57) ABSTRACT

An Embodiment relates to a method for identifying intravascular particles using acoustic analysis that can improve the detection accuracy of circulating tumor cells by capturing particles in blood flowing through a blood vessel at the center of an ultrasonic vortex and identifying the type of the particles in real time.

### CROSS REFERENCE TO RELATED APPLICATION

The present application claims priority to Korean Patent Application No. 10-2024-0110092, filed on Aug. 16, 2024, the entire contents of which are incorporated herein for all purposes by this reference.

### BACKGROUND

### Technical Field

The present disclosure relates to a method for identifying intravascular particles using acoustic analysis that can capture particles in the blood flowing through a blood vessel at the center of an ultrasonic vortex and identify the type of the particles in real time.

### Description of the Related Art

A Circulating Tumor Cell (CTC) is a tumor cell separated from a primary cancer and circulating through blood vessels, and it is a main cause of metastasis to other organs. The survival rate of metastatic cancer caused by circulating tumor cells is very low, and accordingly, technology capable of accurately identifying CTCs is essential to improve patient survival rates before and after cancer treatment.

Blood diagnostic tests that are commonly performed to detect CTCs extract blood and preprocess it, then determine the presence or absence of CTCs in the preprocessed blood. For this purpose, a process of separating intravascular particles by size by generating a standing wave in the blood must be performed in advance.

However, as illustrated in FIG. 1, in order to generate a standing wave, multiple ultrasonic waves are superimposed at both sides opposing each other around the channel through which blood flows, and for this, multiple ultrasound generators are installed to face each other, which results in difficulties in operating and installing the ultrasound generators.

Further, because the number of CTCs is very small compared to other particles, there is a frequent occurrence where no CTC is found in blood samples, depending on the time the blood is extracted, so there is a defect that the reliability is low. Accordingly, considering the above points, there is a demand for technology that can easily operate and install ultrasound generators to separate intravascular particles by size and increase the accuracy of CTC detection in the separated particles.

### SUMMARY

An objective of the present disclosure is to capture intravascular particles flowing through blood vessels at the center of an ultrasonic vortex and to identify the type of the particles in real time.

The objectives of the present disclosure are not limited to those described above and other objectives and advantages not stated herein may be understood through the following description and may be clear by embodiments of the present disclosure. Further, it would be easily known that the objectives and advantages of the present disclosure may be achieved by the configurations described in claims and combinations thereof.

A method for identifying intravascular particles using acoustic analysis according to an embodiment of the present disclosure includes: aligning intravascular particles flowing through a blood vessel in accordance with their sizes by generating a standing wave effect that forms a pressure gradient in the blood vessel; capturing particles at a vortex center by forming an ultrasound vortex in the aligned blood; emitting a diagnostic laser to the vortex center; receiving a response signal to the diagnostic laser; and identifying the type of the captured particles on the basis of intensity of the response signal.

The present disclosure has an effect that it can detect circulating tumor cells in real time by capturing intravascular particles flowing through a blood vessel and identifying their types, and can kill circulating tumor cells immediately upon detection.

Detailed effects of the present disclosure in addition to the above effects will be described with the following detailed description for accomplishing the present disclosure.

### BRIEF DESCRIPTION OF THE DRAWINGS

FIG. 1 is a diagram illustrating an example of multiple ultrasound generators installed to face each other in a channel to generate a standing wave in the related art;

FIG. 2 is a diagram illustrating a CTC capture process according to an embodiment of the present disclosure;

FIG. 3 is a diagram illustrating an apparatus for identifying intravascular particles according to an embodiment of the present disclosure;

FIG. 4 is a flowchart illustrating a CTC detection process;

FIG. 5 is a diagram illustrating intravascular particles being aligned in accordance with their sizes;

FIG. 6 is a simulation image showing pressure nodes and anti-pressure nodes formed in blood due to the effect of a standing wave;

FIG. 7 is an ultrasound image showing intravascular particles aligned in accordance with their sizes;

FIG. 8 is a diagram illustrating intravascular particles captured at the center of an ultrasonic vortex;

FIG. 9 is a diagram illustrating the generation of a photoacoustic signal from a particle to which a diagnostic laser is emitted; and

FIG. 10 is a diagram illustrating emission of a treatment laser in accordance with CTC detection.

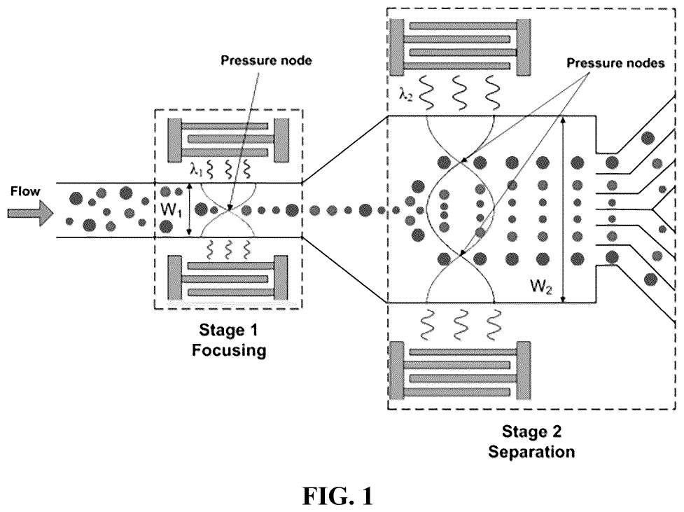

### DETAILED DESCRIPTION

The objectives, characteristics, and advantages will be described in detail below with reference to the accompanying drawings, so those skilled in the art may easily achieve the spirit of the present disclosure. However, in describing the present disclosure, detailed descriptions of well-known technologies will be omitted so as not to obscure the description of the present disclosure with unnecessary details. Hereinafter, exemplary embodiments of the present disclosure are described in detail with reference to the accompanying drawings. The same reference numerals are used to indicate the same or similar components in the drawings.

Although terms ‘first’, ‘second’, etc. are used to describe various components in the specification, it should be noted that these components are not limited by the terms. These terms are used to discriminate one component from another component and it is apparent that a first component may be a second component unless specifically stated otherwise.

Further, in the specification, when a certain configuration is disposed “over (or under)” or “on (beneath)” of a component in the following description, it may mean not only that the certain configuration is disposed on the top (or bottom) of the component, but that another configuration may be interposed between the component and the certain configuration disposed on (or beneath) the component.

Further, in the specification, when a certain component is “connected”, “coupled”, or “jointed” to another component, it should be understood that the components may be directly connected or jointed to each other, but another component may be “interposed” between the components or the components may be “connected”, “coupled”, or “jointed” through another component.

Further, singular forms that are used in this specification are intended to include plural forms unless the context clearly indicates otherwise. In this application, terms “configured”, “include”, or the like should not be construed as necessarily including several components or several steps described herein, in which some of the components or steps may not be included or additional components or steps may be further included.

Further, in this specification, the term “A and/or B” stated in the specification means that A, B, or A and B unless specifically stated otherwise, and the term “C to D” means that C or more and D or less unless specifically stated otherwise.

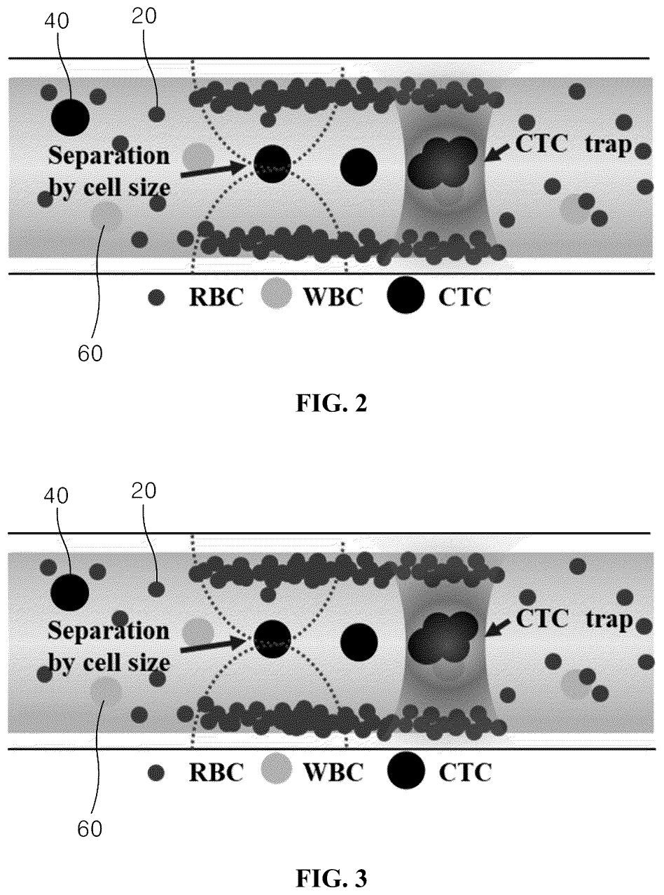

Referring to FIG. 2, an apparatus for identifying intravascular particles using acoustic analysis and a method for identifying particles using the same according to an embodiment of the present disclosure capture particles at the center of an ultrasonic vortex in blood and identify the type of the particles in real time. The particles that are the identification targets may include arbitrary particles flowing in blood.

However, in the following description, in order to describe a specific embodiment of the present disclosure, it is assumed that the particles are circulating tumor cells (CTC) flowing in blood. Accordingly, the present disclosure can be described as an invention that aligns red blood cells 20, white blood cells 60, and circulating tumor cells 40 (CTC), which are intravascular particles flowing through a blood vessel, in accordance with their sizes using a standing wave effect, and captures the circulating tumor cells 40 at the center of an ultrasonic vortex formed in the blood to identify the type of particles. Meanwhile, the standing wave effect may form a pressure gradient in a blood vessel.

In this regard, the apparatus for identifying intravascular particles using acoustic analysis will first be briefly described, and then the method for identifying particle using the apparatus for identifying intravascular particles will be described in detail.

Referring to FIG. 3, an apparatus for identifying intravascular particles may include a case 20, an ultrasound generator 100, a vortex generator 200, a laser generator 300, a transducer 400, and a processor (not shown). However, the apparatus for identifying intravascular particles shown in FIG. 3 is merely one embodiment, and components may be added, changed, or omitted as needed.

The ultrasound generator 100 can output and apply ultrasonic waves into blood. To this end, the ultrasound generator 100 may be installed on the bottom surface inside the case 20 to be adjacent to one side of a blood vessel.

The ultrasound generator 100 can generate a standing wave effect in blood by crossing a plurality of ultrasonic waves output to one side. To this end, the ultrasound generator 100 may include a first ultrasound generator 110 and a second ultrasound generator 120. The first and second ultrasound generators 110 and 120 may be installed at spaced-apart positions inside the case 20 such that the ultrasonic waves output therefrom cross each other at different angles.

The vortex generator 200 can form an ultrasonic vortex in blood in a blood vessel by outputting ultrasonic waves. To this end, the vortex generator 200 may be installed on one inner side of the case 20 to be spaced apart from the ultrasound generator 100, and may be formed in a ring shape to which a plurality of holographic lenses 205 is coupled.

The laser generator 300 can output diagnostic and therapeutic lasers toward the center of a vortex. For this purpose, a diagnostic laser may be installed above the vortex generator 200 to correspond to the center of the ring-shaped vortex generator 200. Accordingly, the diagnostic and therapeutic lasers can pass through the center of the ring shape.

The transducer 400 can receive a response signal to a diagnostic laser. Meanwhile, the transducer 400 may be installed at the center of the ring-shaped vortex generator 200, and the material of the transducer 400 may be transparent. Accordingly, the laser output from the laser generator 300 can pass through the transducer 400 and penetrate into a blood vessel.

The processor can control each component 100, 200, 300, and 400, and accordingly, can identify the type of particles through this. In order to perform this operation, the processor may include at least one physical element among application specific integrated circuits (ASICs), digital signal processors (DSPs), digital signal processing devices (DSPDs), programmable logic devices (PLDs), field programmable gate arrays (FPGAs), controllers, and micro-controllers.

Previously, the apparatus for identifying intravascular particles using acoustic analysis according to an embodiment of the present disclosure was described, and the following describes the operation of the processor for identifying particles using the identification apparatus.

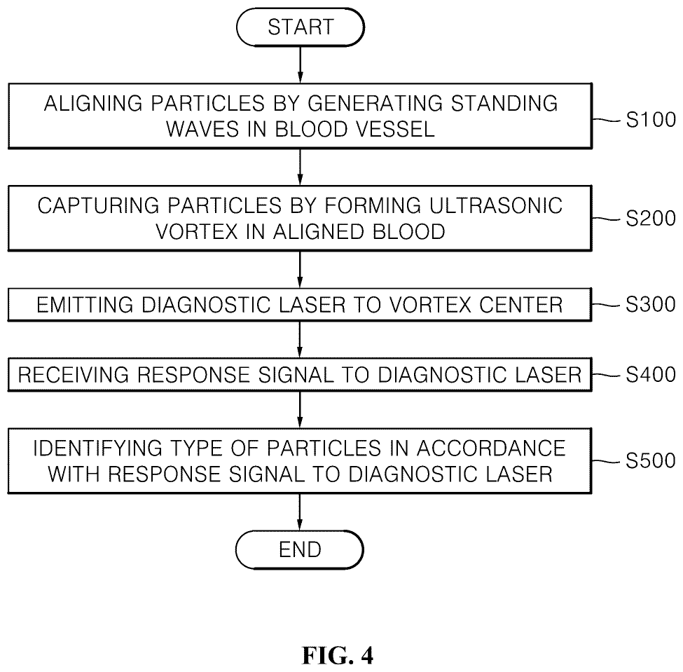

Referring to FIG. 4, a method for identifying intravascular particles using acoustic analysis according to an embodiment of the present disclosure may include: a step of aligning particles by generating a standing wave effect in a blood vessel (S100); a step of capturing particles by forming an ultrasonic vortex in the aligned blood (S200); a step of emitting a diagnostic laser to the center of the vortex (S300); a step of receiving a response signal to the diagnostic laser (S400); and a step of identifying the type of particles on the basis of the response signal. However, the method for identifying particles illustrated in FIG. 4 is merely one embodiment, and some steps may be added, changed, or omitted as needed.

Hereafter, the steps shown in FIG. 4 are described in detail.

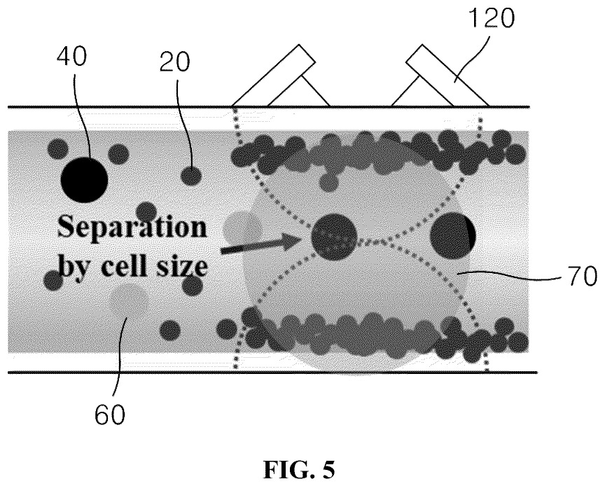

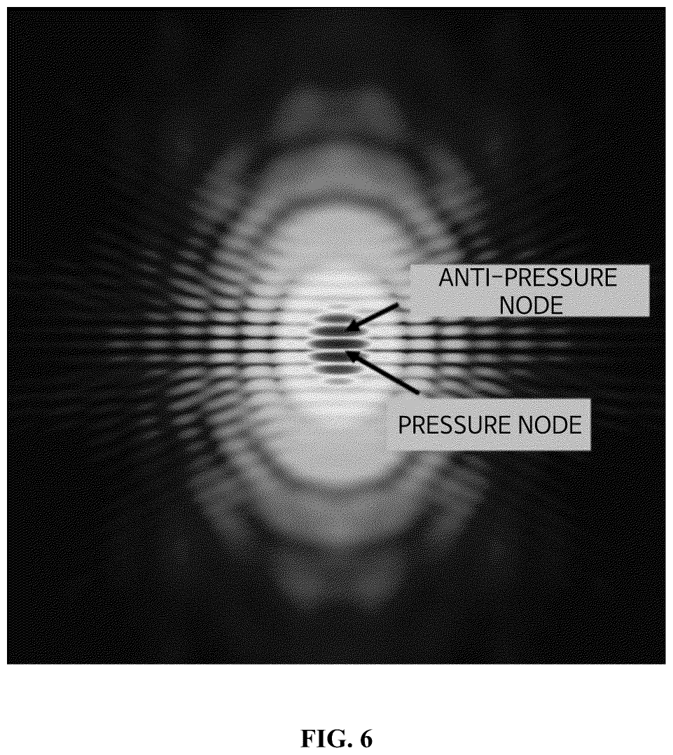

Referring to FIG. 5, the processor can align intravascular particles flowing through a blood vessel in accordance with their sizes by generating a standing wave effect in the blood vessel (S100). Specifically, the processor can generate a standing wave effect that forms a pressure gradient in a blood vessel by controlling the ultrasound generator 100 to generate ultrasonic waves toward one side of the blood vessel.

A plurality of ultrasonic waves generated from the first and second ultrasound generators 110 and 120 may overlap each other, and a standing wave effect can be generated by the overlapped ultrasonic waves.

Specifically, as shown in FIG. 6, a plurality of ultrasonic waves may intersect at different angles. Unlike a plurality of ultrasonic waves that intersects along a X-axis direction and a Z-axis direction in the related art, the standing wave effect may be generated as a plurality of ultrasonic waves output from the X-axis and Y-axis directions overlap in an ‘X’ shape. Accordingly, pressure nodes and anti-pressure nodes may be formed perpendicular to the flow direction of blood in a blood vessel. In this case, the pressure nodes may be formed at the center of the blood vessel, and the anti-pressure nodes may be formed on the inner wall side of the blood vessel. The pressure nodes may have relatively low pressure compared to the anti-pressure nodes.

Accordingly, intravascular particles can move to and align at the pressure nodes or anti-pressure nodes by size. Specifically, larger particles may be aligned closer to the pressure nodes, while smaller particles may be aligned closer to the anti-pressure nodes.

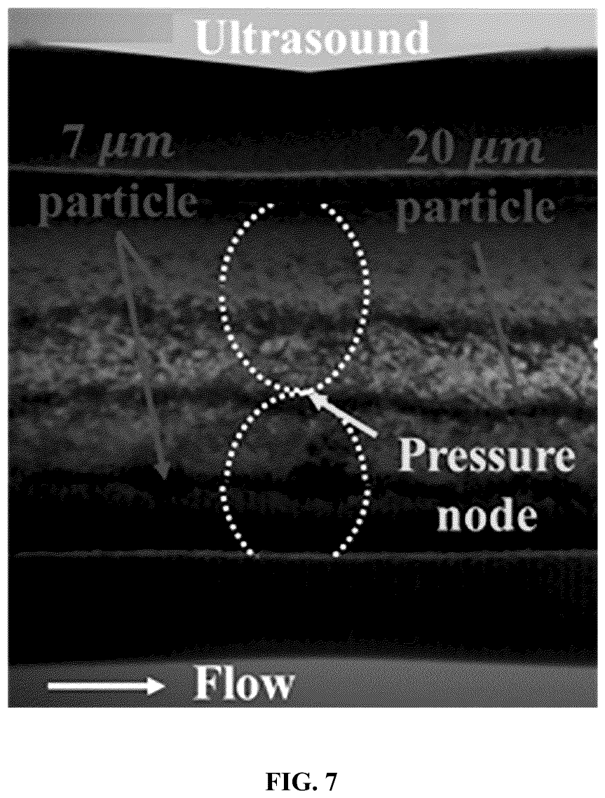

As shown in FIG. 7, relatively small red blood cells 20 (7 μm particles) in blood can be aligned at the anti-pressure nodes, and relatively large white blood cells 60 and circulating tumor cells (20 μm particles) can be aligned at the pressure nodes and gradually flow along the blood.

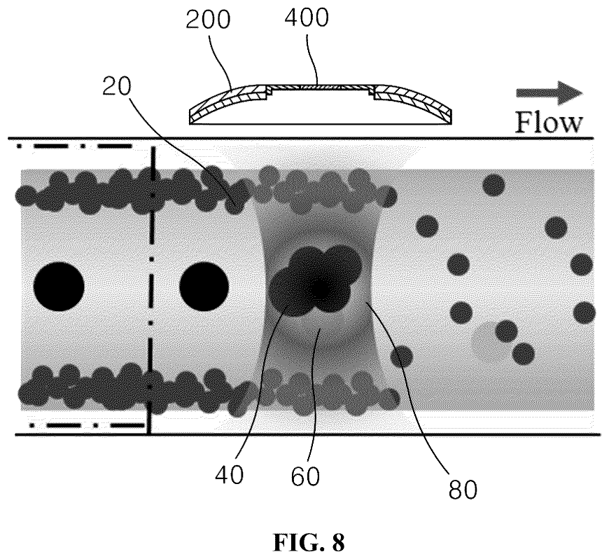

Next, referring to FIG. 8, the processor can form an ultrasonic vortex in the aligned blood and capture particles at the center of the vortex 80 (S200). Specifically, the processor can control the vortex generator 200 to form an ultrasonic vortex inside a blood vessel by outputting ultrasonic waves in a ring shape on one side of the blood vessel.

According to the acoustic radiation force generated by the ultrasonic vortex, particles in the blood vessel can gradually move to the center 80 of the ultrasonic vortex, which is a low-pressure region. However, particles located at the anti-pressure nodes (for example, red blood cells 20) experience negligible acoustic radiation force, so even if these particles enter the ultrasonic vortex, they do not move to the center of the vortex and can move along the direction of blood flow.

On the other hand, particles located at the pressure nodes (for example, white blood cells 60 and circulating tumor cells 40) experience a large acoustic radiation force, so these particles can be captured at the center 80 of the ultrasonic vortex and may no longer flow along with the blood.

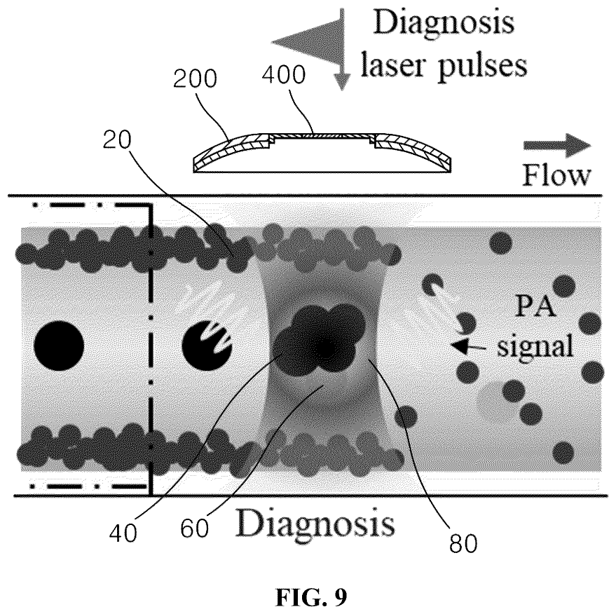

Next, referring to FIG. 9, the processor can emit a diagnostic laser to the center of the ultrasound vortex 80 by using the laser generator 300 (S300).

To this end, the processor can control the laser generator 300 to emit a laser through the transducer 400. That is, when the laser generator 300 is controlled to emit a laser toward the center of the ring-shaped vortex generator 200, the laser can be emitted to the center 80 of the ultrasound vortex through the transparent transducer 400 installed at the center of the ring shape.

Particles captured at the vortex center 80 can generate a response signal (PA signal) by responding to a diagnostic laser of a specific wavelength, for example, a photoacoustic signal.

Next, the processor can receive the response signal (S400). In this case, the intensity of the received response signal may vary depending on the type of particles. The response signal may be stronger as the optical absorption coefficient of particles increases. In one example, the circulating tumor cells 40 in blood have a higher optical absorption coefficient than the white blood cells 60, so the intensity of the response signal may be higher than that of the white blood cells 60.

Next, the processor can identify the type of a captured particle on the basis of the intensity of the response signal generated from the captured particle (S500). Specifically, if the intensity of the response signal is equal to or greater than a threshold, the processor can determine the particle as a circulating tumor cell 40; if the intensity of the response signal is less than the threshold, it can determine it as a white blood cell 60.

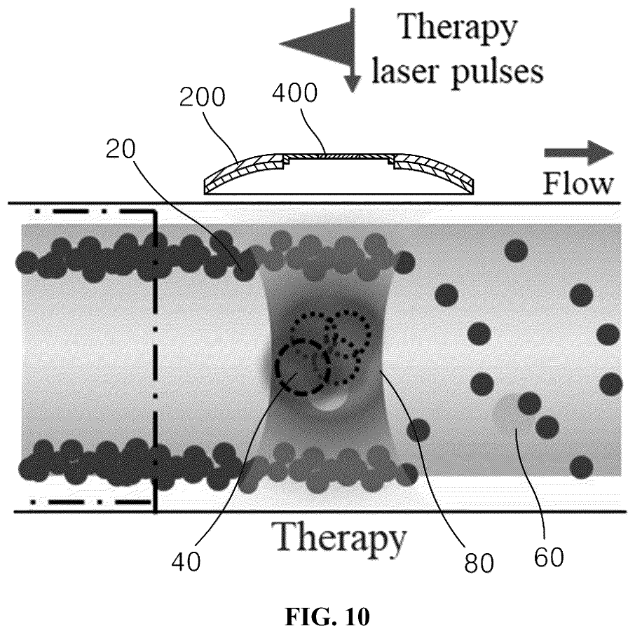

Next, referring to FIG. 10, when the particle type is identified as a circulating tumor cell 40, the processor can output a therapeutic laser to the center 80 of the ultrasound vortex. Specifically, the processor can control the laser generator 300 to output a therapeutic laser to the center 80 of the ultrasound vortex, and in this case, the therapeutic laser may be a high-intensity focused ultrasound (HIFU) capable of destroying the particles captured at the vortex center 80. Accordingly, the circulating tumor cells 40 captured at the vortex center 80 in the example may be killed by the therapeutic laser.

The present disclosure has an effect that it can detect circulating tumor cells in real time by capturing intravascular particles flowing through a blood vessel and identifying their types, and can kill circulating tumor cells immediately upon detection.

Although the present disclosure was described above with reference to the exemplary drawings, it is apparent that the present disclosure is not limited to the embodiments and drawings in the specification and may be modified in various ways by those skilled in the art within the range of the spirit of the present disclosure. Further, even though the operation effects according to the configuration of the present disclosure were not clearly described with the above description of embodiments of the present disclosure, it is apparent that effects that can be expected from the configuration should be also admitted.

What is claimed is:

1. A method for identifying intravascular particles using acoustic analysis, the method comprising:
aligning intravascular particles flowing through a blood vessel in accordance with their sizes by generating a standing wave effect that forms a pressure gradient in the blood vessel by means of a processor;
capturing particles at a vortex center by forming an ultrasound vortex in the aligned blood by means of the processor;
emitting a diagnostic laser to the vortex center by means of the processor;
receiving a response signal to the diagnostic laser by means of the processor; and
identifying the type of the captured particles on the basis of intensity of the response signal by means of the processor.

2. The method of claim 1, wherein the aligning includes generating a standing wave effect that forms the pressure gradient in the blood vessel on one side of the blood vessel.

3. The method of claim 1, wherein the aligning includes generating a standing wave effect by superimposing a plurality of ultrasonic waves.

4. The method of claim 3, wherein the aligning includes generating the standing wave effect by intersecting the plurality of ultrasonic waves at different angles.

5. The method of claim 1, wherein the intravascular particles are aligned by size in accordance with the pressure gradient formed by the standing wave effect.

6. The method of claim 1, wherein pressure nodes and anti-pressure nodes are formed perpendicular to a flow direction of blood in the blood vessel, and
the intravascular particles are aligned such that the larger the size, the closer the intravascular particles are to the pressure nodes, and the smaller the size, the closer the intravascular particles are to the anti-pressure nodes.

7. The method of claim 1, wherein the capturing includes generating the ultrasonic vortex on one side of the blood vessel.

8. The method of claim 1, wherein the capturing includes generating the ultrasonic vortex by outputting ultrasonic waves in a ring shape.

9. The method of claim 1, wherein the capturing includes capturing the particles in accordance with an acoustic radiation force generated by the ultrasonic vortex.

10. The method of claim 1, wherein the particles captured at the vortex center do not move along with the blood.

11. The method of claim 8, wherein the emitting of a diagnostic wave includes passing a diagnostic laser through a center of the ring.

12. The method of claim 1, wherein the receiving of a response signal includes receiving the response signal generated by the captured particles in response to the diagnostic laser.

13. The method of claim 1, wherein the response signal is a photoacoustic signal generated by a particle upon emission of the diagnostic laser.

14. The method of claim 1, wherein the type of the particles includes a white blood cell and a circulating tumor cell (CTC).

15. The method of claim 14, wherein the identifying includes determining that a particle is a circulating tumor cell when intensity of the response signal is equal to or larger than a threshold.

16. The method of claim 15, wherein emitting a therapeutic laser to the vortex center when the type of a particle is identified as a circulating tumor cell.

17. An apparatus for identifying intravascular particles using acoustic analysis, the apparatus comprising:
an ultrasound generator configured to align intravascular particles flowing through a blood vessel in accordance with their sizes by generating a standing wave effect that forms a pressure gradient in the blood vessel;
an ultrasonic vortex generator configured to capture particles at a vortex center by forming an ultrasound vortex in the aligned blood;
a laser generator configured to emit diagnostic and therapeutic lasers to the vortex center;
a transducer configured to receive a response signal to the diagnostic laser; and
a processor configured to identify the type of the captured particles on the basis of intensity of the response signal.

18. The apparatus of claim 17, wherein the ultrasound generator includes:
a first ultrasound generator; and
a second ultrasound generator installed at a position spaced apart from the first ultrasound generator to interest ultrasonic waves generated from the first ultrasound generator at different angles.

19. The apparatus of claim 17, wherein the vortex generator is formed in a ring shape.
